(19) United States
(12) Patent Application Publication
(10) Pub. No.: US 2020/0306562 A1
(43) Pub. Date: Oct. 1, 2020

(54) COMPACT ROTATIONAL GANTRY FOR PROTON RADIATION SYSTEMS

(71) Applicant: Varian Medical Systems Particle Therapy GmbH, Troisdorf (DE)

(72) Inventors: Arno GODEKE, Bonn (DE); Juergen HEESE, Cologne (DE); Michael Schillo, Bonn (DE)

(21) Appl. No.: 16/370,145

(22) Filed: Mar. 29, 2019

(51) Int. Cl. A61N 5/10; H05H 13/00; H05H 7/04

(52) U.S. Cl. CPC A61N 5/1081; A61N 5/1045; H05H 2007/048; H05H 7/04; A61N 2005/1087; H05H 13/005

(57) ABSTRACT

Embodiments of the present invention provide a rotational gantry designed to provide proton radiation therapy using a mono-energetic proton beam. The mono-energetic proton beam is transported by a beam line transport system having two or more bending magnets and a plurality of quadrupole and steerer magnets for directing and focusing the proton beam. Energy variation of the beam is performed directly before the beam reaches an isocenter of the gantry.

### FIELD

Embodiments of the present invention generally relate to the field of particle therapy. More specifically, embodiments of the present invention relate to compact gantries used for particle therapy treatment systems.

### BACKGROUND

To provide proton or particle therapy treatment to a patient, charged particles are directed to the patient on a treatment table at a chosen angle. A gantry including a beamline and bending magnets are used to bring the charged particle beam to the selected angle relative to the patient table. The charged particles are output from an accelerator and emitted into the gantry. Gantries used for particle therapy typically include normal-conducting magnets for bending the particle beam, which requires a gantry having a diameter on the order of 8 meters. Furthermore, the substantial weight of the bending magnets must be supported by the mechanical structure of the gantry.

Moreover, the energy of the particle beam must be adjusted by introducing variable thickness wedges into the beam path. This is typically done before the beam enters the gantry. The wedges will also spread the particle beam due to multiple scattering effects. Any multitude of particles produced by an accelerator (e.g., a beam) typically have a slight variation of energies between individual particles. The energy spread is the statistically correct derived amount of energy variation around the median energy value of this beam. In order to transport the degraded beam through the gantry to the patient, a large transversal aperture inside the magnets is needed because of the beam divergence and energy distribution around the median energy. The large aperture further increases the size and weight of the magnets.

Additionally, a large fraction of the beam is stopped before the gantry when varying the beam energy causing the beam to diverge and spreading the beam energy in the process, leading to the need for large magnet apertures. This leads to reduced beam efficiency, and these large high-energy beam losses also create the need for substantial concrete shielding, which also increases the building costs substantially. Another main disadvantage of existing proton beamlines is the small beam transmission between the particle accelerator and the isocenter of the gantry. At low energies, up to 99.5% of the beam is stopped in the degrader section, thus requiring a relatively high accelerator output current and significant radiation shielding walls in the accelerator and degrader areas. Again, this leads to increased building costs and size.

Furthermore, existing particle therapy gantries change energy levels between treatment depths in the patient. To accommodate these fast energy changes of the particle beam, the bending magnets must rapidly alter their magnetic field amplitude. This fast ramping of the bending magnets generates various electrodynamic losses in the magnet conductor and other electrically conducting elements, which, in the case of superconducting magnets, leads to the risk of local hot-spots that can trigger a rapid transition in the magnet to the normal conducting state (“magnet quench”). To mitigate these ramping losses, a need has emerged for high cooling capacity and low-loss conductors with, in the case of superconductors, a small filament size. An alternative solution is the use of achromatic bending magnets that can accommodate a large range of energies of the particle beam; however, this solution is very expensive and requires large bore dipole or combined function magnets in combination with high quadrupole magnetic fields to re-focus the beam. These large components lead to large gantry designs and structures.

### SUMMARY OF THE INVENTION

Embodiments of the present invention provide a rotational gantry designed to provide proton radiation therapy using a mono-energetic proton beam. The mono-energetic proton beam is transported by a beam line transport system having two or more bending magnets and a plurality of quadrupole and steerer magnets for directing and focusing the proton beam. Energy variation of the beam is performed directly before the beam reaches an isocenter of the gantry.

According to one embodiment, a rotational gantry for a proton radiation system is disclosed. The gantry includes an entry point operable to receive a mono-energetic proton beam from an accelerator, a beam line transport system including two or more bending magnets comprising at least a first bend magnet and a final bend magnet, where the final bend magnet is disposed at a position corresponding to a final bend of the gantry, and a plurality of quadrupole and steerer magnets operable to direct and focus the mono-energetic proton beam. The gantry further includes a two-dimensional beam spreading system positioned downstream from the final bend magnet, and an energy varying component positioned downstream from the beam spreading system, the energy varying component operable to receive the mono-energetic proton beam and for varying an energy thereof before reaching an isocenter of the gantry.

According to another embodiment, a gantry for a proton radiation therapy system is disclosed. The gantry includes a physical containment and supporting structure including a receiver side and an emitter side, where the receiver side is operable to receive a proton beam emitted from an accelerator, where the proton beam is compact and mono-energetic, a plurality of small bore, fixed field, beam bending magnets disposed within the physical containment and supporting structure, where the plurality of small bore, fixed field, bending magnets include a first magnet disposed proximate to the receiver side and operable to bend the proton beam by a first degree amount, and a second magnet disposed proximate to the emitter side and operable to bend the proton beam by a second degree amount and through the emitter side and towards an isocenter of the gantry, where the second magnet includes a superconducting magnet, and a plurality of small aperture beamline magnets disposed within the physical containment and supporting structure, the plurality of small aperture beamline magnets including a plurality of steerer magnets, and a plurality of quadrupole magnets disposed between the first and second, or after the second magnet(s).

Yet another embodiment discloses a compact proton radiation therapy system including an accelerator operable to emit a proton beam that is compact and mono-energetic, a gantry coupled to the accelerator and including a physical containment and supporting structure including a receiver side and an emitter side, where the receiver side is operable to receive the proton beam emitted from the accelerator, a plurality of small bore, fixed field, beam bending magnets disposed within the physical containment and supporting structure, where the plurality of small bore, fixed field, bending magnets include a first magnet disposed proximate to the receiver side and operable to bend the proton beam by a first degree amount, and a second magnet disposed proximate to the emitter side and operable to bend the proton beam by a second degree amount through the emitter side and towards an isocenter of the gantry, where the second magnet includes a superconducting magnet, and a plurality of small aperture beamline magnets disposed within the physical containment and supporting structure, the plurality of small aperture beamline magnets including a plurality of steerer magnets, and a plurality of quadrupole magnets disposed between the first and second, or after the second magnet(s), and a degrader disposed on the gantry and operable to receive the proton beam from the second magnet and to vary an energy level thereof, and an XY scanner disposed to receive an output proton beam from the degrader and to generate an output beam to a target.

### BRIEF DESCRIPTION OF THE DRAWINGS

The accompanying drawings, which are incorporated in and form a part of this specification and in which like numerals depict like elements, illustrate embodiments of the present disclosure and, together with the description, serve to explain the principles of the disclosure.

FIG. 1 depicts an exemplary accelerator and rotational gantry including quadrupoles for focusing a mono-energetic proton beam and bending magnets for targeting an isocenter according to embodiments of the present invention.

FIG. 2 depicts an exemplary accelerator and rotational gantry including quadrupoles for focusing a mono-energetic proton beam and bending magnets for targeting an isocenter according to embodiments of the present invention.

FIG. 3 depicts an exemplary accelerator and rotational gantry including quadrupoles for focusing a mono-energetic proton beam and bending magnets for targeting an isocenter according to embodiments of the present invention.

FIG. 4 depicts an exemplary proton radiation system having multiple rotational gantries according to embodiments of the present invention.

### DETAILED DESCRIPTION

Reference will now be made in detail to several embodiments. While the subject matter will be described in conjunction with the alternative embodiments, it will be understood that they are not intended to limit the claimed subject matter to these embodiments. On the contrary, the claimed subject matter is intended to cover alternative, modifications, and equivalents, which may be included within the spirit and scope of the claimed subject matter as defined by the appended claims.

Furthermore, in the following detailed description, numerous specific details are set forth in order to provide a thorough understanding of the claimed subject matter. However, it will be recognized by one skilled in the art that embodiments may be practiced without these specific details or with equivalents thereof. In other instances, well-known methods, procedures, components, and circuits have not been described in detail as not to unnecessarily obscure aspects and features of the subject matter.

### Fixed-Energy Rotational Gantry with Superconducting Bending Magnets

Embodiments of the present invention provide a compact gantry designed to provide particle therapy using a compact mono-energetic beam. The components that perform energy variation of the beam are moved to a position directly before the patient, and do not require rapid alterations to the magnetic field within the gantry. The use of the compact mono-energetic beam allows the gantry to advantageously utilize relatively small-bore bending magnets (e.g., superconducting bending magnets), and the bending magnets can be produced at a relatively low cost compared to existing conducting solutions. Varying energy before the patient in this way (and not before or in the gantry) eliminates most of the beam losses and enables the use of a limited aperture for the magnets. Moreover, by using small-bore, fixed field superconducting bending magnets, the gantry can use a very compact and simple magnet design with relatively low weight, and the costs associated with transportation and on-site installation of the gantry are significantly reduced. The bore of a magnet refers to a central opening in the magnet were a beam can pass through. The dimensions of this opening strongly correlate to the complexity, weight, and size of the magnet. The dimension of the bore is typically chosen to be as small as possible to pass the beam through without incurring a loss of particles, which is determined based on the emittance of the beam.

A degrader or range shifter may be included to vary the energy of a proton beam using a scattering material of varying thickness in the beam path. By using a fixed energy transport system, embodiments of the present invention can avoid large beam losses after a separate degrader section before the gantry, and a much larger fraction of the particles coming out of the accelerator can therefore reach the patient. In this way, the treatment system only needs to deliver the protons that are actually used for patient treatment. Furthermore, the required shielding walls can be significantly reduced as there are no degrader sections with high beam losses. For example, according to some embodiments, radiation shielding is only used to shield radiation produced by the treatment (e.g., protons stopped in a patient).

The superconducting bending magnets described herein may have an available open aperture as small as 20 mm, for example. According to some embodiments, the last bending magnet of the gantry can be a simple dipole or combined function magnet, and the bending radius for a fixed energy output approximately between 100 MeV and 250 MeV protons can be 30 cm when the magnet is superconducting with a dipole field of 7.7 T. A magnetic field amplitude of 7.7 T is achievable using conventional low temperature superconducting technology. Similar magnetic fields can also be achieved using high temperature superconductors with significant temperature margins, thereby simplifying the cooling requirements. Also combined function magnets can be considered, as well as a combination of normal conducting and superconducting coil sections within a magnet. The magnets can furthermore be actively and/or shielded to reduce the stray magnetic fields, or passively shielded according to some embodiments.

Furthermore, by including an optimized scanning nozzle, the gantry, including the superconducting bending magnets, can be as small as 2.0 m in radius, whereas radii of around 3.0 m are achievable using normal conducting bending magnets. The size of the scanning nozzle directly impacts the outer diameter requirement of the gantry. For example, the source-to-axis distance (SAD) representing the distance from the middle of the scanner to the isocenter may be reduced to a minimum of 1 m. According to some embodiments, the first element directly after the bending magnet is a compact combined XY scanner 60 cm in length. An XY scanner is an electromagnetic device that bends a beam in two orthogonal directions (e.g., X and Y) that are perpendicular to the beam direction. The XY scanner may include a sequence of two bending magnets (e.g., one for X direction and one for Y direction) which the beam passes or a combination of two dipole or combined function magnets in one place, etc. A multi-strip dose and position ionization chamber may be positioned after the scanner to monitoring of the actual delivered dose and pencil beam position.

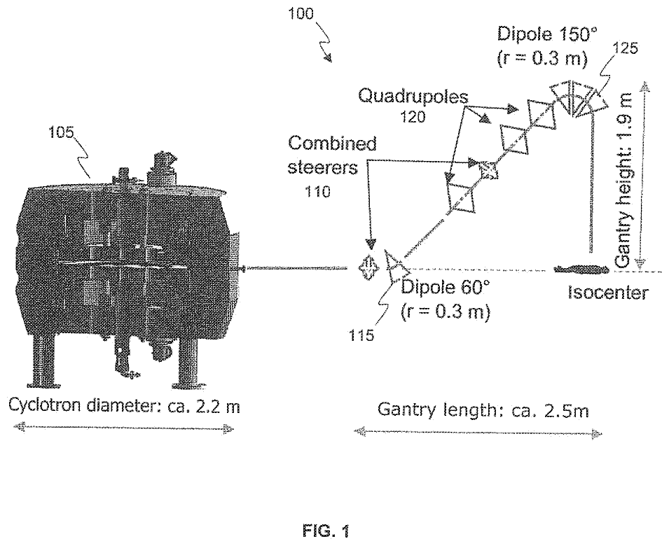

With regard to FIG. 1, an exemplary compact mono-energetic gantry 100 including a superconducting bending magnet 125 is depicted according to embodiments of the present invention. Superconducting magnets are generally far lighter than comparable conventional magnets and smaller radii can be achieved since the attainable magnetic fields for bending the particles can be higher. The inclusion of a superconducting bending magnet also enables less physical space to be required to accommodate the beam therapy system. Compared to existing gantries that use conventional magnets that transport beams of varying energy, the volume of gantry 100 can be reduced by a factor of 20, the length of gantry 100 is approximately 2.5 m or less, and the height of the gantry 100 is approximately 1.9 m or less in the case of a superconducting final bending magnet, and approximately 3.0 m or less in the case of a normal conducting final bending magnet. The bending magnets described according to the various embodiments of the present invention may include one of, or a combination of dipoles (e.g., high field dipoles), combined function magnets, superconducting magnets, and normal conducting magnets. These magnets can be passively or actively shielded according to various embodiments.

In the example of FIG. 1, gantry 100 includes a first bending dipole or combined function 115 having an angle of approximately 60 degrees for bending the mono-energetic beam produced by accelerator 105 (e.g., a cyclotron). One of ordinary skill in the art will recognize that conventional accelerators can be used to produce the mono-energetic beam within the scope of the various embodiments described herein.

The gantry is supported by a physical containment and supporting structure (not pictured) having an emitter side that emits a charged particle beam and a receiver side operable to receive the charged particle beam produced by the accelerator. A set of combined steerers 110 shift the beam in a direction without focusing the beam

The upward portion of the beamline includes multiple (e.g., three) small quadrupole magnets 120 to focus the beam by creating a negative dispersion to compensate for the natural beam dispersion and the dispersion in the last bend caused by superconducting bending magnet 125. The beamline components used to implement gantry 100 can be relatively small in size due to the small size of the mono-energetic beam generated by the accelerator. The magnetic field used to guide the beam can remain unchanged during treatment and does not require multiple ramping stages. However, a specific ramping speed may be required for initial ramp-up, maintenance, and recovery, for example, or if two or more different mono-energetic beam energy levels are desired. The protons exiting the accelerator are at an energy between 100 and 250 MeV.

The last bending magnet includes a superconducting bending magnet 125 having a bending angle of approximately 150 degrees. According to some embodiments, the superconducting bending magnet 125 includes two bent racetrack coils in between which a dipole magnetic field is generated. More advanced and/or efficient magnet designs can be employed. For example, high temperature superconductors capable of generating approximately 7.7 T at an operating temperature of approximately 10 K may be used, where the cryocoolers (not shown) used to cool the high temperature superconductors are one order of magnitude more effective than when operating at temperatures of approximately 4 K. According to some embodiments, 150-degree magnet 125 and 60-degree magnet 115 both include superconducting magnets. Furthermore, according to some embodiments, the magnet 115 has an angle approximately between 45 degrees and 60 degrees, and the magnet 125 has an angle approximately between 135 degrees and 150 degrees.

Starting from an open bore diameter of 20 mm, and accommodating a coil support structure and cryostat, the inner diameter of the windings of bending magnet 125 may be approximately 50 mm diameter, leading to a required outer diameter of 125 mm at a typical 300 A/mm2, according to some embodiments. The additional conductor cost for high temperature superconductors can be compensated for using significantly simpler coil manufacturing and cooling. It is appreciated that either low- or high temperature superconductors, or a combination of both, can be used.

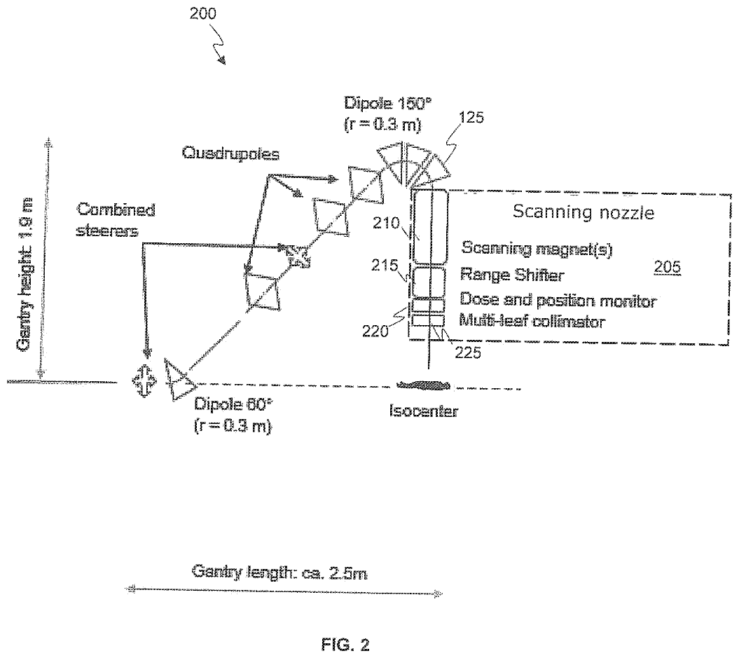

With regard to FIG. 2, an exemplary gantry 200 including a superconducting bending magnet is depicted according to embodiments of the present invention. While gantry 200 is similar to gantry 100 depicted in FIG. 1, gantry 200 includes a scanning nozzle 205 configured to direct the beam to an isocenter (e.g., target) using one or more scanning magnets 210, a range shifter 215, a dose and position monitor 220, and a multi-leaf collimator 225.

Following 150-degree dipole or combined function magnet 125, scanning magnets 210 output a charged particle beam which is directed to range shifter 215 which modulates the energy of the beam. For example, scanning magnets 210 may be configured to scan the beam in both horizontal and vertical directions and form an irradiation field of a specific shape and size. Range shifter 215 may be an energy variation system and include stopping material for reducing the residual range of the particle beam such that the treatment ranges can be adjusted to a target depth. According to some embodiments, multiple plates made of appropriate material (e.g. polycarbonate, carbon, etc.) are included to adapt the energy of the protons to a specified level. The 150 degree dipole can be a high field dipole in some embodiments.

The output of range shifter 215 is received by a dose and position monitor 220 to monitor the actual delivered dose and beam position. For example, the dose and position monitor 220 includes a multi-strip dose and position ionization chamber, according to some embodiments. The dose and position monitor 220 is followed by a multi-leaf collimator that sharpens the outer contours According to some embodiments, the radius of the gantry 200 is 3 m or less. According to some embodiments, the length of the gantry 200 is 3 m or less. Furthermore, according to some embodiments, the magnet 125 has an angle approximately between 135 degrees and 150 degrees. One of ordinary skill in the art will recognize that conventional scanning nozzles can be used to direct the beam to the isocenter using typical scanning magnets, range shifters, dose and position monitors, multi-leaf collimators within the scope of the embodiment depicted in FIG. 2.

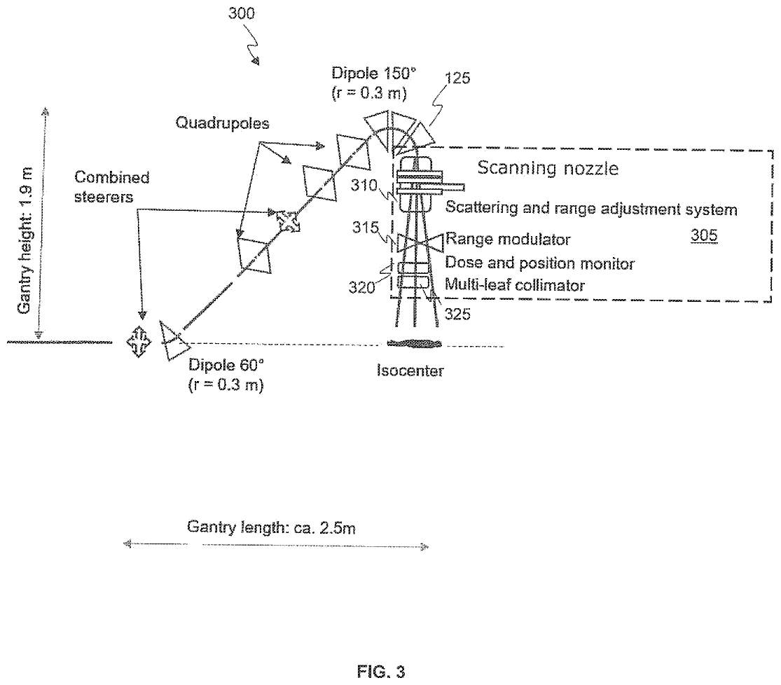

With regard to FIG. 3, an exemplary gantry 300 including a superconducting bending magnet is depicted according to embodiments of the present invention. While gantry 300 is similar to gantry 100 depicted in FIG. 1, gantry 300 includes a scattering nozzle 305 configured to direct the beam to an isocenter (e.g., target) using a scattering and range adjustment system 310, a range modulator 315, a dose and position monitor 320, and a multi-leaf collimator 325.

Following 150-degree dipole or combined function magnet 125, scattering and range adjustment system 310 outputs a charged particle beam which is directed to range modulator 315. For example, scattering and range adjustment system 310 may be configured to spread the beam in both horizontal and vertical directions and form an irradiation field of a specific shape and size. The output of range modulator 315 is received by a dose and position monitor 320 to monitor the actual delivered dose and beam position. For example, the dose and position monitor 320 includes a multi-strip dose and position ionization chamber, according to some embodiments. The dose and position monitor 320 is followed by a multi-leaf collimator that sharpens the outer contours of the beam at relatively low energy levels. According to some embodiments, the magnet 125 has an angle approximately between 135 degrees and 150 degrees. One of ordinary skill in the art will recognize that conventional scanning nozzles can be used to direct the beam to the isocenter using typical scattering and range adjustment systems, range modulators, dose and position monitors, and multi-leaf collimators within the scope of the embodiment depicted in FIG. 3.

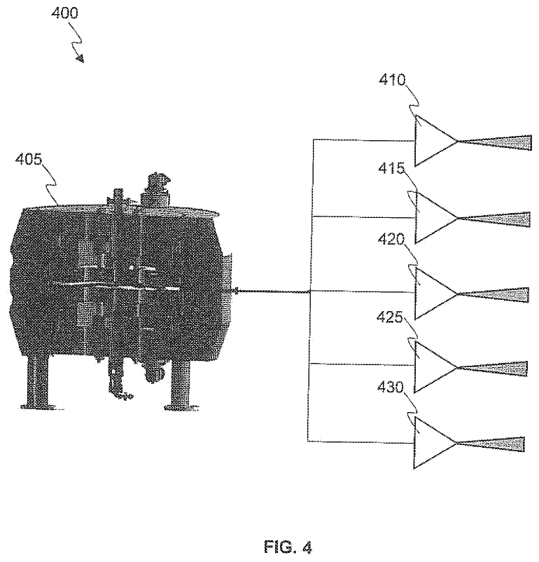

With regard to FIG. 4, an exemplary multi-gantry beam (e.g., proton radiation) therapy treatment system 400 is depicted according to embodiments of the present invention. Individual gantries 410-430 may be installed in separate areas or rooms of a beam therapy treatment center, for example. Accelerator 405 generates a mono-energetic beam with low emittance. This beam is guided through a beamline consisting of a multitude of quadrupole, bending and steerer magnets into a switch yard comprised of quadrupole and steerer magnets including one or more dipole or combined function magnets which can be powered selectively to bend the beam into a selected gantry room. All the beamline components and the magnets of gantries 410-430 can be relatively small in size. The magnetic field used to bend the beam in the gantries can remain unchanged during treatment and does not require multiple ramping stages. The protons exiting the accelerator 405 can be in an energy range between 100 to 250 MeV

The gantries 410-430 may include a superconducting bending magnet which enables higher magnetic fields and smaller radii, and less physical space is required to accommodate the beam therapy system. Compared to existing gantries that use conventional magnets, the volume of the gantries 410-430 can be reduced by a factor of 20 such that the length of the gantries 410-430 is approximately 2.5 m or less, and the height of the gantries 410-430 is approximately 1.9 m or less when using superconducting final bending magnets, and 3.0 m or less when using normal conducting final bending magnets. Multi-room or single-room gantries may be used in accordance with any of the embodiments of the present invention.

The gantries 410-430 may have a scanning nozzle including a two-dimensional beam spreading system, for example, a lateral beam spreading system, and may include a range shifter comprising a plurality of plates made of polycarbonate or carbon. The scanning nozzle may further include a multi-strip dose and position ionization chamber disposed after an XY scanner that monitors the actual dose delivered and the beam position of particle beam. Because the energy variation of beam is performed directly before the beam reaches the target, the gantries 410-430 can be designed to accommodate mono-energetic compact beams. In this way, the diameter of the gantries 410-430 can be reduced to around 3 m for normal conducting bend magnets and around 2 m or less for superconducting bend magnets.

According to one embodiment, a rotational gantry for a proton radiation system is disclosed. The gantry includes an entry point operable to receive a mono-energetic proton beam from an accelerator, a beam line transport system including two or more bending magnets comprising at least a first bend magnet and a final bend magnet, where the final bend magnet is disposed at a position corresponding to a final bend of the gantry, and a plurality of quadrupole and steerer magnets operable to direct and focus the mono-energetic proton beam. The gantry further includes a two dimensional beam spreading system positioned downstream from the final bend magnet, and an energy varying component positioned downstream from the beam spreading system, the energy varying component operable to receive the mono-energetic proton beam and for varying an energy thereof before reaching an isocenter of the gantry.

According to another embodiment, a gantry for a proton radiation therapy system is disclosed. The gantry includes a physical containment and supporting structure including a receiver side and an emitter side, where the receiver side is operable to receive a proton beam emitted from an accelerator, where the proton beam is compact and mono-energetic, a plurality of small bore, fixed field, beam bending magnets disposed within the physical containment and supporting structure, where the plurality of small bore, fixed field, bending magnets include a first magnet disposed proximate to the receiver side and operable to bend the proton beam by a first degree amount, and a second magnet disposed proximate to the emitter side and operable to bend the proton beam by a second degree amount and through the emitter side and towards an isocenter of the gantry, where the second magnet includes a superconducting magnet, and a plurality of small aperture beamline magnets disposed within the physical containment and supporting structure, the plurality of small aperture beamline magnets including a plurality of steerer magnets, and a plurality of quadrupole magnets disposed between the first and second, or after the second magnet(s).

Yet another embodiment discloses a compact proton radiation therapy system including an accelerator operable to emit a proton beam that is compact and mono-energetic, a gantry coupled to the accelerator and including a physical containment and supporting structure including a receiver side and an emitter side, where the receiver side is operable to receive the proton beam emitted from the accelerator, a plurality of small bore, fixed field, beam bending magnets disposed within the physical containment and supporting structure, where the plurality of small bore, fixed field, bending magnets include a first magnet disposed proximate to the receiver side and operable to bend the proton beam by a first degree amount, and a second magnet disposed proximate to the emitter side and operable to bend the proton beam by a second degree amount through the emitter side and towards an isocenter of the gantry, where the second magnet includes a superconducting magnet, and a plurality of small aperture beamline magnets disposed within the physical containment and supporting structure, the plurality of small aperture beamline magnets including a plurality of steerer magnets, and a plurality of quadrupole magnets disposed between the first and second, or after the second magnet(s), and a degrader disposed on the gantry and operable to receive the proton beam from the second magnet and to vary an energy level thereof, and an XY scanner disposed to receive an output proton beam from the degrader and to generate an output beam to a target.

According to one embodiment, the two or more bending magnets include one of, or a combination of a dipole, a combined function magnet, a normal conducting magnet, and a superconducting magnet.

According to one embodiment, the first degree-amount is approximately between 45 and 60 degrees and the second degree-amount is approximately between 135 and 150 degrees.

According to one embodiment, the first magnet includes a bend radius of 0.3 meters, the second magnet include a bend radius of approximately 0.3 meters, and the second magnet is a superconducting magnet.

According to one embodiment, the gantry has a gantry radius of approximately 1.9 meters or less and a gantry length of approximately 2.5 meters or less.

According to one embodiment, the plurality of quadrupole magnets includes a first quadrupole magnet, a second quadrupole magnet, and a third quadrupole magnet, where the second steerer magnet is disposed between the first and second quadrupole magnets.

According to one embodiment, the first, second, and third quadrupole magnets each have an open bore diameter of 20 mm.

According to one embodiment, the mono-energetic proton beam is approximately between 100 MeV and 250 MeV.

According to one embodiment, the second magnet includes a bending radius of 30 cm and produces a dipole field of 7.7 T.

According to one embodiment, the second magnet includes an open bore diameter of 20 mm, an inner diameter of windings of 50 mm, and an outer diameter of the windings of 125 mm.

According to one embodiment, the radiation therapy system further includes a multi-strip dose and position ionization chamber disposed to receive the output beam from the XY scanner, a range shifter, and a multi-leaf collimator disposed after the range shifter.

According to one embodiment, the first magnet and the second magnet include one of, or a combination of: a dipole; a combined function magnet; a normal conducting magnet; and a superconducting magnet.

Embodiments of the present invention are thus described. While the present invention has been described in particular embodiments, it should be appreciated that the present invention should not be construed as limited by such embodiments, but rather construed according to the following claims.

1. A rotational gantry for a proton radiation system, said gantry comprising:
an entry point operable to receive a mono-energetic proton beam from an accelerator;
a beam line transport system comprising:
two or more bending magnets comprising at least a first bend magnet and a final bend magnet, wherein said final bend magnet is disposed at a position corresponding to a final bend of said gantry, and wherein said final bend magnet comprises a superconducting magnet having an open bore diameter of approximately 20 mm; and
a plurality of quadrupole and steerer magnets operable to direct and focus said mono-energetic proton beam;
a two-dimensional beam spreading system positioned downstream from said final bend magnet; and
an energy varying component positioned downstream from said beam spreading system, said energy varying component operable to receive said mono-energetic proton beam and for varying an energy thereof before reaching an isocenter of said gantry.

2. The gantry as described in claim 1 wherein said beam line transport system is characterized as transporting a proton beam of a fixed energy and a small aperture.

3. The gantry as described in claim 2 wherein said mono-energetic beam is between 100 MeV and 250 MeV.

4. The gantry as described in claim 1 wherein said two or more bending magnets comprise one of, or a combination of: a superconducting magnet; a normal conducting magnet; a dipole; and a combined function magnet.

5. The gantry as described in claim 1 wherein said two or more bending magnets comprise superconducting magnets, and wherein said superconducting magnets comprise one of, or a combination of: LTS superconductor material and HTS superconductor material.

6. The gantry as described in claim 1 having a gantry radius of less than 3 meters.

7. The gantry as described in claim 1 having a gantry length of less than 3 meters.

8. The gantry as described in claim 1 wherein said accelerator is a cyclotron.

9. The gantry as described in claim 1 comprising a plurality of bends and wherein a first bend is approximately between 45 and 60 degrees and wherein said final bend is approximately between 135 and 150 degrees.

10. A proton radiation system comprising:
an accelerator operable to provide a mono-energetic proton beam; and
an isocentric rotational gantry disposed to receive said mono-energetic proton beam from said accelerator and comprising:
a beam line transport system comprising:
two or more bending magnets comprising at least a first bend magnet and a final bend magnet, wherein said final bend magnet is disposed at a position corresponding to a final bend of said gantry, and wherein said final bend magnet comprises a superconducting magnet having an open bore diameter of approximately 20 mm; and
a plurality of quadrupole and steerer magnets operable to direct and focus said mono-energetic proton beam;
a two-dimensional beam spreading system positioned downstream from said final bend magnet; and
an energy varying component positioned downstream from said beam spreading system, said energy varying component operable to receive said mono-energetic proton beam and for varying an energy thereof before reaching an isocenter of said gantry.

11. The system as described in claim 10 wherein said beam line transport system is characterized by a small aperture.

12. The system as described in claim 11 wherein said beam line transport system is characterized as transporting a mono-energetic proton beam.

13. The system as described in claim 12 wherein said mono-energetic beam is between 100 MeV and 250 MeV.

14. The system as described in claim 10 wherein said two or more bending magnets comprise one of, or a combination of: a dipole; a combined function magnet; a normal conducting magnet; and a superconducting magnet.

15. The system as described in claim 10 wherein said two or more bending magnets comprise superconducting magnets, and wherein said superconducting magnets comprise one of, or a combination of: LTS superconductor material and HTS superconductor material.

16. The system as described in claim 10 wherein said gantry has a radius of less than 3 meters and wherein said energy varying component comprises a range shifter.

17. The system as described in claim 10 wherein said gantry has a length of less than 3 meters and wherein said energy varying component comprises a range shifter.

18. The system as described in claim 10 wherein said accelerator is a cyclotron.

19. The system as described in claim 10 wherein said gantry comprises a plurality of bends and wherein a first bend is approximately between 45 and 60 degrees and wherein said final bend is approximately between 135 and 150 degrees.

20. A compact proton radiation therapy system comprising:
an accelerator operable to emit a small aperture mono-energetic proton beam;
a gantry coupled to said accelerator and comprising:
a physical containment and supporting structure comprising a receiver side and an emitter side, wherein said receiver side is operable to receive said proton beam emitted from said accelerator;
a plurality of small bore, fixed field, beam bending magnets disposed within said gantry and comprising:
a first magnet disposed proximate to said receiver side and operable to bend said proton beam by a first degree amount; and
a second magnet disposed proximate to said emitter side and operable to bend said proton beam by a second degree amount through said emitter side and towards an isocenter of said gantry, wherein said second magnet comprises a superconducting magnet having an open bore diameter of approximately 20 mm; and
a plurality of small aperture beamline magnets disposed within said gantry and comprising:
a plurality of steerer magnets; and
a plurality of quadrupole magnets; and
a range shifter disposed on said gantry downstream from a second dipole or combined function magnet and operable to receive said proton beam and to vary an energy level thereof.

21. The system as described in claim 20 further comprising:
an XY scanner operable to generate an output beam to a target;
a multi-strip dose and position ionization chamber disposed to receive said output beam from said XY scanner; and
a multi-leaf collimator disposed downstream of said range shifter.

22. The system as described in claim 20 wherein said first degree amount is approximately between 45 and 60 degrees and wherein said second degree amount is approximately between 135 and 150 degrees.

23. The system as described in claim 20 wherein said first magnet and said second magnet comprise one of, or a combination of: a dipole; a combined function magnet; a normal conducting magnet; and a superconducting magnet.
